(19) United States
(12) Patent Application Publication
(10) Pub. No.: US 2025/0203190 A1
(43) Pub. Date: Jun. 19, 2025

(54) ENDOSCOPE WITH LIGHT GUIDE AND LEDS IN DISTAL TIP

(71) Applicant: AMBU A/S, Ballerup (DK)

(72) Inventor: Morten SØRENSEN, Ballerup (DK)

(73) Assignee: AMBU A/S, Ballerup (DK)

(21) Appl. No.: 18/959,764

(22) Filed: Nov. 26, 2024

(30) Foreign Application Priority Data
Dec. 19, 2023 (EP) 23218067.9

(51) Int. Cl. H04N 23/55; H04N 23/50; H04N 23/51; H04N 23/56; H04N 23/57

(52) U.S. Cl. CPC H04N 23/55; H04N 23/51; H04N 23/555; H04N 23/56; H04N 23/57

(57) ABSTRACT

An endoscope (1) with a distal tip (10), where the distal tip comprises a camera (11) and a light emitter (13). The light emitter includes a first LED (15) configured to emit light with a first spectral distribution, and a second LED (15′) configured to emit light with a second spectral distribution, different from the first spectral distribution. The light emitter includes a light guide (30) with a length, and a width transverse to the length. The light guide includes a light entry end (31) and a light exit (32) end configured such that light passes the light guide in the length direction. The light emitter also includes a diffuser (34). The first and the second LEDs are arranged to emit light into the light entry end, and the diffuser is arranged such that light passing the light exit end also passes through the diffuser.

### CROSS-REFERENCE TO RELATED APPLICATIONS

This application claims priority from and the benefit of European Patent Application No. 23218067.9, filed Dec. 19, 2023; the disclosure of said application is incorporated by reference herein in its entirety.

### TECHNICAL FIELD

The present teaching relates to an endoscope comprising a light emitter in a distal tip. The light emitter comprises diodes having different spectral distributions.

### BACKGROUND

In endoscopy for medical purposes endoscopes are used for visual navigation into, and examination and diagnosis of, hollow organs and body cavities. For such purposes the quality of the image provided to the endoscope's user is an important parameter. The level of this quality is primarily influenced by the camera and the light provided to the target to be observed. Both the amount of light and the distribution of light in the image field of view is important for the image quality.

When applying endoscopes in medical procedures it is, for some procedures, preferred to have the option of changing the spectral distribution of the light during the procedure. This is typically the case when specific types of tissue or characteristics in the tissue are to be enhanced. Such image enhancement can be done by different application of fluorescence techniques, or by application of light having a spectral distribution with peaks at wavelengths where tissue or cells are known to have a high absorbance. For example, it is possible to enhance image contrast of the vascular areas relative to surroundings, by providing peaks of light intensity at wavelengths where hemoglobin has a high absorbance.

To enable changing between a white light mode and a mode with colored light having peaks at specific wavelengths, it is necessary to be able to change between different spectral distributions of the light during a procedure. This can be done by having at least two different light sources available and being able to turn these on and off separately. The light sources may be arranged in a special box connected to the endoscope and the light emitted from the light sources guided via optical fibers through the endoscope to the distal tip thereof.

For single-use endoscopes, which are designed to be used for only one procedure and then discarded, it may be preferred to have the light source built into the distal tip of the endoscope, whereby optical fibers going from the handle of the endoscope to the distal tip can be avoided, thereby facilitating simpler and less costly manufacturing.

When viewing tissue with an endoscope, direct reflections of the light from the light source will often be visible in the image, especially when surfaces with a high specular reflectance, such as humid or wet surfaces, are viewed. Users of endoscopes will be familiar with this and will ignore it. However, if the white light is formed from two or more light sources having different colors, there may be a separate reflection of each light source having the color of this light source. This may be distracting for the endoscope user.

### SUMMARY

A first aspect of the disclosure provides an endoscope comprising a distal tip having a light emitter and a camera, where the light emitter comprises Light Emitting Diodes (LEDs) having different spectral distributions and a light guide.

A second aspect of the present disclosure relates to a system comprising an endoscope according to the first aspect and variations thereof, a monitor and a control unit.

In one embodiment according to the first aspect, the endoscope comprises, in the distal tip, a light emitter and a camera with an optical axis. The light emitter comprises a first LED configured to emit light with a first spectral distribution and a second LED configured to emit light with a second spectral distribution which is different from the first spectral distribution. Further, the light emitter comprises a light guide having a length, a width transverse to the length, a light entry end, and a light exit end configured such that light passes the light guide in the length direction. The light emitter also comprises a diffuser arranged transverse to the optical axis. The first and the second LEDs are arranged to emit light into the light entry end, and the diffuser is arranged such that light passing the light exit end also passes the diffuser. The length direction may be parallel to the optical axis.

It has been found that the diffuser according to this embodiment can provide a better mixing of different colored light into one combined spectrum, and thereby reduce colored reflections considerably, or even eliminate such reflections. This is achieved by an improved mixing of light from the first LED and the second LED. Also, the diffuser according to this embodiment achieves a very homogenous distribution of light, such that the image quality of the endoscope is improved.

Further features and combinations consistent with the present disclosure will become apparent the following detailed description. Features described in the present disclosure can be combined so as to form further arrangements within the scope of the present disclosure that are not explicitly set out herein.

### BRIEF DESCRIPTION OF THE FIGURES

The above-mentioned embodiments, features and advantages thereof will be further elucidated by the following illustrative and nonlimiting detailed description of embodiments disclosed herein with reference to the appended drawings, wherein:

FIG. 1 shows a visualization system comprising an endoscope and a monitor with a control unit.

FIG. 2 shows a tip housing seen from a distal end.

FIG. 3 shows a tip housing connected to a bending section.

FIG. 4 shows schematic view of a camera.

FIG. 5 shows a tip housing seen from a proximal end.

FIG. 6 shows a perspective view of a light emitter.

FIG. 7 illustrates the intensity distribution of light exiting a light guide having a length of 1 mm.

FIG. 8 illustrates the intensity distribution of light exiting a light guide having a length of 2 mm.

FIG. 9 illustrates the intensity distribution of light exiting a light guide having a length of 4 mm.

FIG. 10 illustrates the intensity distribution of light exiting a light guide having a length of 6 mm.

In the drawings, corresponding reference characters indicate corresponding parts, functions, and features throughout the several views. The drawings are not necessarily to scale and certain features may be exaggerated in order to better illustrate and explain the disclosed embodiments. For simplicity, this disclosure will focus on a two-way bending endoscope, but the disclosure is relevant for, and covers, also a four-way bending endoscope.

### DETAILED DESCRIPTION

The disclosures of the following patents and/or publications are incorporated by reference herein: commonly-owned U.S. Pat. No. 11,291,352, commonly-owned U.S. Pat. No. 11,766,163, and commonly-owned U.S. Pat. No. 12,053,152.

The term “distal” means in the direction away from the user of the endoscope and toward the patient, and the term “proximal” means in the direction toward the endoscope's user. Thus, a proximal end of a component is closer to the user than a distal end of the component. For the handle of the endoscope, the distal end will be the end where the main tube is connected, and the proximal end is the opposite end.

The term “handle” may be a positioning interface, or interface, which functions to control the position of the insertion cord. The handle, or positioning interface, may be an interface operated by a robotic arm, or it may be a handle operated by the hand of an endoscope's user.

Endoscopes include procedure-specialized endoscopes, such as arthroscopes, bronchoscopes, colonoscopes, cystoscopes, duodenoscopes, ENT scopes, gastroscopes, ureteroscopes, and any other device comprising an interface connected to an insertion cord comprising a camera.

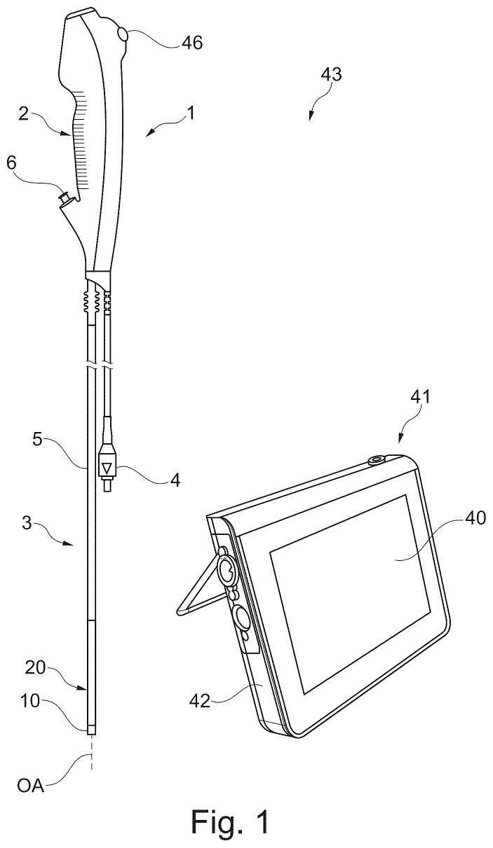

FIG. 1 illustrates a visualization system 43 comprising an endoscope 1 and a monitor 41. The endoscope comprises a handle 2, an insertion cord 3 and an electrical cable with a connector 4 for connecting the endoscope 1 to the monitor 41. The insertion cord 3 is the part to be inserted into a body lumen during an endoscopic procedure. The insertion cord 3 comprises a distal tip 10, a bending section 20 and a main tube 5. The handle 2 may comprise an entrance to a working channel 6 running through the insertion cord 3 to an opening at the distal tip 10. The handle also comprises a bending lever 46, which can be used for bending the bending section 20.

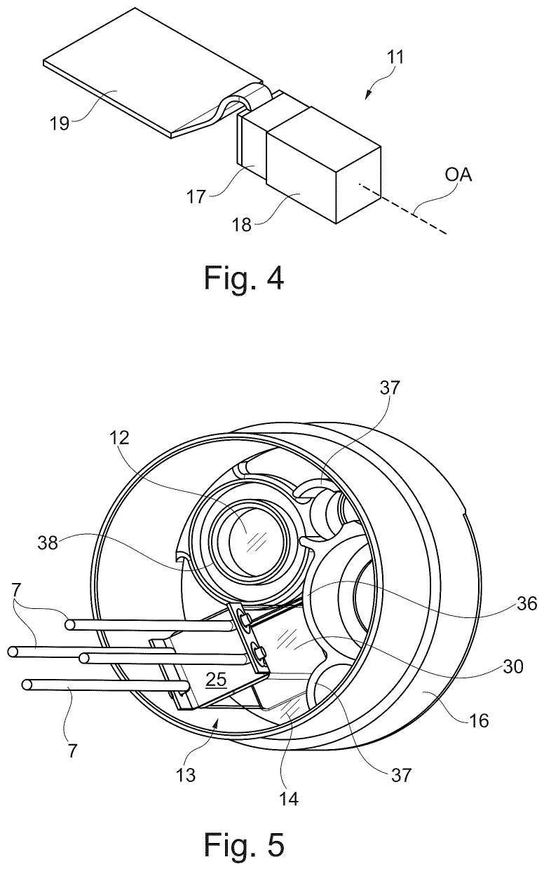

The monitor 41 comprises a screen 40 and a control unit 42. The control unit 42 comprises an electronic circuit for receiving and processing the image stream from the camera 11 (see FIG. 4) as well as a processor for image processing, user interface, storage of images etc. The screen 40 displays the image stream from the camera 11. The monitor 41 may comprise a housing at least partially surrounding the screen 40 and the control unit 42. The screen 40 and the control unit 42 may also be separate parts, e.g. each part comprising a housing at least partially surrounding, respectively, the screen 40 and the control unit 42.

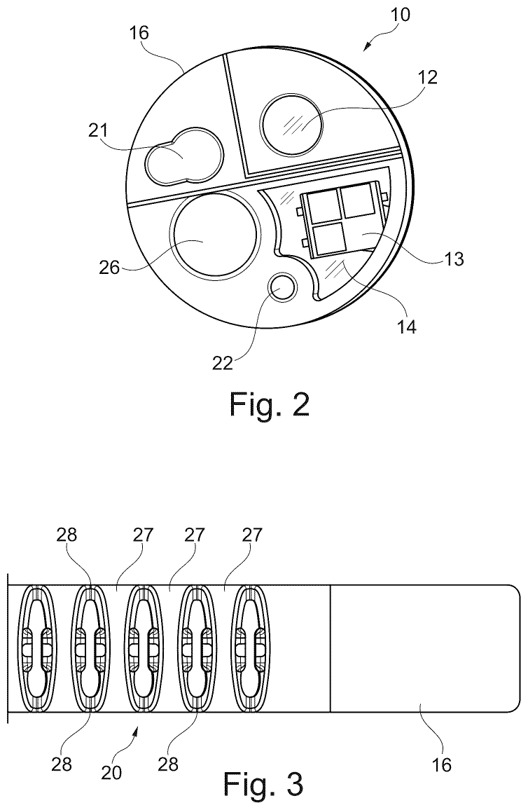

FIG. 2 shows an example of the distal tip 10 of the endoscope 1 shown in FIG. 1 viewed from a distal end. The distal tip 10 comprises a tip housing 16 including a camera window 12, a light emitter window 14, a cleaning nozzle 21, a waterjet nozzle 22, and a distal working channel opening 26. Instruments can be provided, e.g., for taking tissue samples, through the distal working channel opening 26. The cleaning nozzle 21 is provided for cleaning the outside of the camera window 12 by irrigation of cleansing water. The waterjet nozzle 22 is provided for possible cleaning of any tissue to be studied by use of the endoscope. A light emitter 13 is shown, positioned proximally of the light emitter window 14, and is described below. In another example, the distal tip 10 is devoid of the cleaning nozzle 21 and a waterjet nozzle 22. In a further example, the endoscope is devoid of the cleaning nozzle 21, the waterjet nozzle 22, and the working channel.

As shown in FIG. 3, the tip housing 16 may be connected to a distal end of the bending section 20. The bending section 20 may be molded in one piece from a polymer material, such as Polyoxymethylene (POM). Typically, a bending cover (not shown) covers the bending section 20 to make this part watertight and to provide a smooth outer surface. In this example the bending section is molded in one piece comprising segments 27 interconnected by hinges 28 to form a one-piece bending section 20. The segment 27 at the proximal end of the bending section 20 is connected to the distal end of the main tube 5 (not shown in FIG. 3). The segments are held together by hinges 28 so that the segments can be bent relative to each other by manipulation of steering wires (not shown) controlled by the bending lever 46.

Referring now to FIG. 4, the distal tip 10 comprises a camera 11 having an image sensor 17 and a lens stack 18. The camera 11 is placed proximally and adjacent the camera window 12 and is arranged to view tissue, or any observation target which is found relevant to study during a procedure, on the external side of the endoscope's distal tip. The camera 11 has an optical axis OA which may be coincident with a center axis of the lens stack 18. The optical axis OA may extend perpendicular to a plane parallel with an imaging plane in which pixels of the image sensor 17 lie. A flexible printed circuit board 19 may connect the image sensor 17 to wires (not shown) which are connected to a circuit board in the handle or to the control unit 42. The center of the image sensor 17 may here be defined as the center of the part of the image sensor 17 forming images to be analyzed and presented to a user of the endoscope 1. The optical axis OA may extend in parallel with a longitudinal axis extending in a distal to proximal direction of the distal tip 10. The optical axis OA may extend through the center of the image sensor 17 and the center of the imaged part of the target to be observed.

The camera 11 will have a field of view, measured as an angle, defined by the image sensor 17 and the lens stack 18. Images or a stream of images captured by the camera 11 may be shown on the monitor 41, e.g., after image processing.

FIG. 5 shows the tip housing 16 viewed from a proximal end in a perspective view. As shown in FIG. 5, the camera 11 may be arranged inside the tip housing 16. A camera frame (not shown) made of a polymer material may support the flexible printed circuit board 19. The tip housing 16 may be molded from a polymer material, e.g., from a thermoplastic polymer, such as polycarbonate (PC) or COC, COP, PMMA. Also, silicone may be applied for the tip housing. The tip housing 16 may be molded in a two-component molding process, from a transparent material and a non-transparent material, respectively. The transparent material may preferably be applied for the camera window 12 and light emitter window 14. The non-transparent material may be applied for the rest of the tip housing 16. By applying the two-component molding process, the transparent and non-transparent parts of the housing will be fused together in a strong and watertight connection. Also, a process of connecting two separate housing parts, e.g., by gluing, can be avoided. This may simplify manufacturing. Also, the application of non-transparent material for parts of the housing, other than the windows, reduces the risk of stray-light into the camera. However, the tip housing may comprise transparent portions provided to allow UV curing of adhesives inside the tip housing. A tip housing and a method of manufacturing the tip housing, e.g. tip housing 16, are disclosed in commonly-owned U.S. Pat. No. 11,291,352 and commonly-owned U.S. Pat. No. 12,053,152.

Inside the tip housing 16, internal structures may be provided. Such internal structures are seen in FIG. 5 and may be molded as part of the tip housing 16. This internal structure may comprise a support wall 36 for the working channel tubing and support walls 37 for the media tubes providing water for the cleaning nozzle 21 or waterjet nozzle 22. The internal structure may further comprise support structure 38 for supporting and aligning the camera 11. Such support structure 38 for the camera may be a wall encircling the distal part of the camera. Such a wall, made from a non-transparent material, may support the positioning of the camera 11 and may also prevent stray-light from an LED, potentially an LED of the light emitter, passing into the camera. Support walls 36, 37 and support structure 38 enable a simple and fast assembly of the distal tip 10 providing stable and watertight connections.

In an example, the distal tip comprises the housing 16 with the support 38 for positioning the camera 11, where the support 38 is non-transparent to prevent entry of stray-light into the camera 11 or affecting images captured by the image sensor 17. As any stray-light could originate from one LED alone, this feature further reduces the risk of having parts of the image from the endoscope unintentionally colored.

As illustrated in FIGS. 2 and 5, an example of a light emitter 13 is arranged in the tip housing 16. The light emitter 13 is arranged to emit light towards an observation target through the light emitter window 14 (see FIG. 2). The light emitter window 14 may be separated from the camera window 12 by a non-transparent part of a distal wall of the distal tip to minimize risk of direct light reflections into the camera 11 via one common window in front of both camera 11 and light emitter 13. The the camera window 12 and the light emitter window 14 may also be part of the distal wall of the distal tip. The distal wall has an interior, or proximal, side and an exterior, or distal, side. Cables 17 are shown extending proximally from the light emitter 25. Generally, a cable is a conductor surrounded by insulation. The conductor may comprise one or more wires.

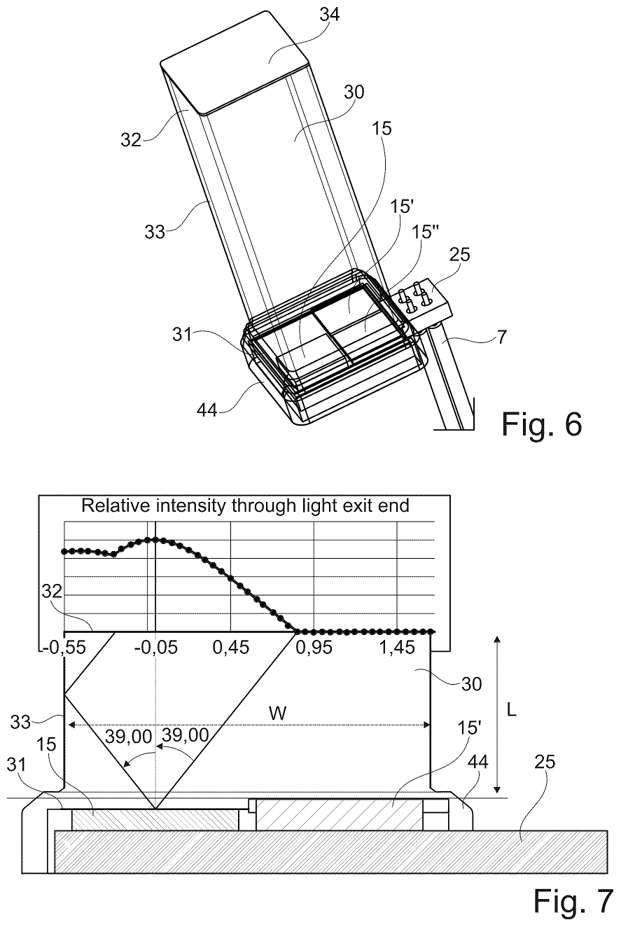

An example of a light emitter 13 is shown in FIG. 6. The light emitter in FIG. 6 comprises a light guide 30 having a light entry end 31 and a light exit end 32. The light emitter is configured such that light passes through the light guide in the length direction. LEDs 15, 15′, 15″ are arranged at the light entry end 31 to emit light into the light entry end of the light guide 30. The light entry end 31 may be a plane or a substantially plane surface, and the at least two LEDs 15 may be arranged with the light emitting part/surface of the LEDs next to this surface. The light emitting surfaces may abut or be contiguous with the light entry end 31. Alternatively, a thin layer of adhesive may be placed therebetween. In any case, it is prefered that there be no air gap between the light emitting surfaces and the light entry end 31. The number of LEDs may be two, three or four. The LEDs may emit light having different spectral distributions. At least two LEDs can emit light having different spectral distributions. However, each LED may emit light with a different spectral distribution. The LEDs may be connected to a printed circuit board (PCB) 25 through which power is provided to the LEDs. The PCB 25 may be a rigid PCB, enabling simpler alignment of the LEDs during manufacturing, or it may be a flexible PCB, e.g., a flex-print. An optional diffuser 34, explained below, is also shown. Side walls 33 connect the light entry end 31 and the light exit end 32.

Alternatively, the LEDs may be staggered longitudinally to allow an overall reduction in cross-section. This may be possible when the light emitting surface of the LED is smaller than its periphery. Thus, one LED may be positioned distally, and overlapping, the periphery of another, but not its light emitting surface.

Alternatively, rather than using multiple independently packaged LEDs, two or more LEDs can be manufactured in one package, potentially reducing the cross-section of the package relative to multiple packages, and simplifying electrical connections.

The LEDs often have a peak in light intensity at a certain wavelength. In an example an LED, such as a first LED, has a peak of light emitted in the range 405-425 nm. In an example an LED, such as a second LED, has a peak of light emitted in the range 520-540 nm. Such peaks in the light are advantageous for improving contrast in vascular tissue, as hemoglobin has peaks of light absorbance in narrow bandwidth wavelengths around 415 nm and 530 nm, where the 415 nm light penetrates only the outer surface layer of the tissue and the 530 nm light can penetrate deeper into the tissue. The combination of light from the two wavelength ranges gives an improved contrast of vascular tissue.

A peak in the spectral distribution of light from an LED is here understood as a local maximum in light intensity at a given wavelength. An LED may have only one peak in its spectral distribution of light. But an LED may also have two or three peaks or more.

The light emitter may comprise a third LED configured to emit light with a third spectral distribution which is different from the first and second spectral distributions. A third LED gives further options for selecting one or more spectral distributions of the light applied during an endoscope procedure. A third LED could have a peak of light emitted in the range 550-670 nm. Mixing especially light above 580 nm with the above suggested ranges for the first and/or second LED enables generation of white light with a good visualization of details in tissue. There may also be a fourth LED, having a peak of light in a fourth wavelength range, e.g., in the range 440-460 nm, or alternatively a fourth LED could have a broad wave length distribution providing a white light.

LEDs can have different beam angles. A narrow beam angle spans between 10-30 degrees, a medium beam angle spans between 30-60 degrees, a wide beam angle spans between 60-120 degrees, and an ultra wide beam angle exceeds 120 degrees.

The LEDs 15 are provided with power through the cables 7. The cables 7 may extend through the insertion cord 3 to the handle 2 or through the handle to the control unit 42. The LEDs may each have one separate cable for the anode, such that the power to each LED can be individually controlled. The separate anode may be connected to each LED through the PCB 25. The LEDs may have one common cathode cable to minimize the number of cables. The common cathode may be connected to each LED through the PCB 25. The individually controllable LEDs enable changes in both color of light and intensity of light during a procedure. This may be applied for different light settings, e.g., pre-programmed, which may be selectable by the user, or automatically adjustable based on analysis of obtained images.

The emitting surfaces of the LEDs 15 may be placed as close to each other as possible to facilitate better mixing of the light from the different LEDs. If three or four LEDs are used, arrangement of the LEDs in a pattern forming a 2×2 matrix may be a way to place them as close as possible. Such a 2×2 matrix can be seen in FIGS. 2 and 6, where the three LEDs are arranged at the light entry end of the light guide, and in this example, they are arranged in a 2×2 matrix. I.e., there will be one empty spot in the matrix.

The light guide 30 may be provided with a rim 44 at the light entry end (see FIG. 6). The rim 44 may encircle the PCB 25 with the LEDs and form an indentation or recess in which the PCB 25 is placed. The shape of the PCB 25 and the shape of the indentation formed by the rim 44, may be configured to fit each other, so that the position of the PCB 25 is aligned in relation to the light guide 30 when the PCB is arranged in the indentation. This facilitates a simple and reliable assembly process for the manufacturing. After placement of the PCB with LEDs, the indentation may be filled with glue to seal and fixate the PCB with LEDs. Part of the PCB may extend outside this sealing and connect to the cables 7 providing power supply to the LEDs.

The light guide 30 may have a rectangular or squared cross-sectional shape when the cross-section is made transversely to a longitudinal axis of the light guide 30, going from a center of the light entry end 31 to a center of the light exit end 32. FIGS. 2, 5, and 6 illustrate a squared cross-sectional shape. The term center may here be understood as a geometric center. This longitudinal axis may be parallel to an optical axis of the camera. The shape could also take any other form, such as circular or pentagonal or any polygon shape. When the shape is rectangular, or any polygon, the longitudinal corners may be rounded, e.g. a rounded rectangle or a rounded polygon. Rounding may be a result of the molting process, in which case the curvature radius is small and the curvature removes the corner edge. An advantage by the light guide 30 having a fully or substantially rectangular or squared cross-sectional shape is that most commercially available LEDs have a rectangular or squared shape, and the endoscope image shown to the user via the monitor 41 is also rectangular or squared.

The cross-sectional area of the light guide 30 may be the same from the light entry end 31 to the light exit end 32. Alternatively, the cross-sectional area may expand so that the cross-sectional-area close to the light exit end 32 is larger than the cross-sectional area close to the light entry end 31. This will have the effect of reducing the spreading of the light, i.e., changing the spatial distribution of emitted light towards a more collimated light beam. However, when a diffuser is added at the light exit end 32, as described below, the diffuser will have the major impact on the spreading of light. One further advantage of the expanding cross-sectional area of the light guide 30, is that if the light guide is integrated with the light emitter window 14, and e.g., molded in one part with this, the demolding will be simpler. The same advantage is present if the light guide is molded as an integrated part of the tip housing 16.

A width of the light guide may be defined as the length of the sides when the cross-section of the light guide, transversely to the longitudinal axis, forms a square. If the cross-section is rectangular or any other shape, the width may be the length of the sides of a square having the same cross-sectional area. A length of the light guide may be defined as the distance from a center of the light entry end 31 to a center of the light exit end 32, i.e., the length along a longitudinal axis of the light guide.

It is desirable to apply the smallest form factor that provides sufficient homogeneous distribution of light, with the smallest LEDs that provide enough light and have the smallest cross-section, so as to reduce the size of the imaging subassembly of the endoscope while enclosing the light guide entirely within the tip housing 16. The imaging subassembly includes the camera, the flexible circuit board, and the light emitter. A small imaging subassembly facilitates a reduced cross-section of the distal tip, which may be smaller than 3.2 mm in diameter, and/or frees up space in the tip housing to allow for larger working channels or the addition of irrigation or other fluid channels. Ideally, the length of the tip housing is defined by the size of camera (lens stack and image sensor) and the flexible circuit board. If the light guide exceeds this length, the tip part may become longer, which impedes navigation via steering of the tip part.

As discussed below in relation to FIGS. 7-9, a longer light guide will result in a better mixing of the light from different LEDs emitting light of different colors. In an example of the light guide dimensions, the width of the light guide 30 may be 1-2.5 mm, and the length of the light guide may be 2-8 mm. These dimentions allow manufacturing of a small diameter distal tip. A small diameter may be, for example, 3.2 mm or less.

In a preferred variation, the light guide 30 has a form factor of at least 2, meaning that the length is at least 2× larger than the width. A form factor of 2 results in a sufficiently homogeneous output beam from LEDs having wide beam angles. This result is surprising, it was not expected that a light guide with such a small form factor could produce a sufficiently homogeneous output beam. In one example, the light guide has a form factor of 2, the LEDs are arranged in a 2×2 matrix, the light guide has a square cross-section, the width of the light guide is less than 2.3 mm, the emitting surfaces of the LEDs fit within the light entry end 31 of the light guide, and the LEDs have wide beam angles. The cross-section may be that of a rounded square. The LEDs may be square with 1×1 mm emitting surfaces. The LEDs may be provided in a single package. In a prefered example, the light guide has a form factor between 2.5 and 3.5. This form factor range is an improvement over a sufficient form factor, as described with reference to FIGS. 7-10 below, and is therefore more desirable while still allowing use of a “short” light guide and distal tip. In this example the LEDs are arranged in a 2×2 matrix, the light guide has a square or rounded square cross-section, the width of the light guide is less than 2.3 mm, and the LEDs are square with 1×1 mm emitting surfaces and wide beam angles, e.g. 120 degrees. The entirety of the emitting surfaces emit into the light entry end 31 of the light guide 30. The light guide may have a 2.2×2.2 cross-section with a length of between 5.5 and 8.0 mm. Of course the form factor can be increased above 3.5 to further improve the homogeneity of the output beam.

In an example, the length of the light guide 30 is at maximum a factor of 8 larger than the width of the light guide, or the length of the light guide 30 is at maximum a factor of 6 larger than the width of the light guide. This will enable for a very good mixing of light in the light guide and will often be short enough to keep the light guide 30 and the LEDs 15 within the tip housing 16.

The distal tip 10 of the endoscope 1 may comprise a light emitter window 14 arranged such that light from the light exit end 32 passes through the light emitter window 14, and where the light emitter window 14 has an inner surface facing the light guide and an oppositely placed external surface. The light guide 30 and the light emitter window 14 may be one integrated part, in which case the inner surface of the light emitter window 14 and the light exit end 32 will be the same plane inside the material forming the integrated light guide 30 and light emitter window 14. This plane may be transverse to the optical direction of the camera 11.

An integrated light guide 30 and light emitter window 14 may be an integrated part of the tip housing 16. The three parts may be fused together, e.g., in a molding process. The molding process may be a two component (or two-shot) molding process, such that both the transparent parts, e.g., camera window 12, light emitter window 14, and light guide 30, and the non-transparent parts, e.g., outer wall of tip housing 16 different from windows, inner support walls 36, 37, and support structure 38 can be formed in one process.

The light guide 30 may be surrounded by air along its side walls 33, to ensure total internal reflection (TIR) to provide the desired illumination characteristics or light distribution profile from the light source. TIR is based on the characteristics of the materials, which define a critical angle. The critical angle is measured from a plane perpendicular to a transition surface between two mediums, one being the material of the light guide and the other being the material or volume surrounding it. If the angle of incidence of light rays emitted by a light source into the light guide is larger than the critical angle, meaning that rays are closer to being parallel to the transition surface, the rays refracted at the transition surface will not emerge from the medium in which they are transmitted but will be reflected back into the medium. The geometry of the light guides 15 are configured, together with the choice of light source and materials, to substantially reduce the likelihood of a light ray impinging on the transition surface at an angle smaller than the critical angle. The configurations may involve index of refraction of the material, transparency, length, variation of the cross-sectional area, geometry of the cross-section, cladding etc.

Generally, to achieve TIR light has to travel from an optically denser medium (higher refractive index) to an optically less dense medium (lower refractive index). If cladding is used, the cladding has to be optically less dense than the material of the light guide, which is why air works well. Example materials for the light guide include acrylic (poly(methyl methacrylate)), polycarbonate, glass, and other polymers.

The light guide 30 is designed to collect the light emitted from the LEDs. The light rays from one LED 15 will either go straight through the light guide from the light entry end 31 to the light exit end 32 without being reflected at any side walls 33 of the light guide, or it will be reflected by side walls 33 a number of times. The LEDs are not centered on the light entry end 31, therefore, more light rays from a single LED will be reflected by the side wall closest to the LED 15. Depending on the length of the light guide 30 the number of light rays being reflected by side walls will increase and at a certain length the light rays will be reflected on both the closest side wall and on the opposite side wall.

This is illustrated in FIGS. 7-10 showing how light from one LED 15 may be distributed by light guides having different length/width (L/W) ratios. These figures show cross-sectional views of the light guide 30 in a plane extending in parallel with the longitudinal axis of the light guide. The cross-sectional view passes two LEDs 15, 15′ and the PCB 25. The difference between FIGS. 7, 8, 9 and 10 is the length L of the light guide 30.

In each of FIGS. 7-10 a graph has been placed at the light exit end 32 surface of the light guide 30. This graph illustrates how light intensity from one LED 15 is distributed in this cross-sectional view coinciding with the light exit end 32. I.e., the graphs show the situation where only one LED 15 is powered to emit light. The data in these graphs are based on calculations where the light rays emitted from the LED with the largest angle of scattering are shown. This angle is here 39 degrees as indicated, but the angle may vary depending on the LEDs applied. The light guide 30 is in the examples shown in FIGS. 7-10 made from polycarbonate. The x-axis of these graphs extends in parallel with the cross-section and perpendicular to the longitudinal axis of the light guide 30. The zero point of the x-axis is aligned with the center of the light emitting area of the active LED 15. The unit for the x-axis is millimeter. The y-axis of the graphs extends in parallel with the longitudinal axis of the light guide 30. The zero point of the y-axis has been placed at the light exit end 32. The shape of each curve illustrates the relative intensity distribution. The numbers indicated on the y-axis are only relative numbers.

As seen from FIGS. 7-10, the distribution of light intensity from one LED 15 across the light exit end 32, gets more homogeneous with increasing length L of the light guide 30. By “more homogeneous” it is meant that the intensity variation is smaller and thus the line in the graph is flatter. Simulations and tests have shown that a light guide 30 with a cross section of approximately 2×2 mm has an approximately fully homogeneous light intensity distribution when it is at least 6 mm long (i.e., a width W to length L ratio of 1:3 or a form factor of 3). The distribution of light intensity for a 6 mm long light guide is shown in FIG. 10. The light intensity values were calculated for a plurality of points on a line at the light exit end 32 which overlaps a center of an LED and is parallel to a side of the light guide.

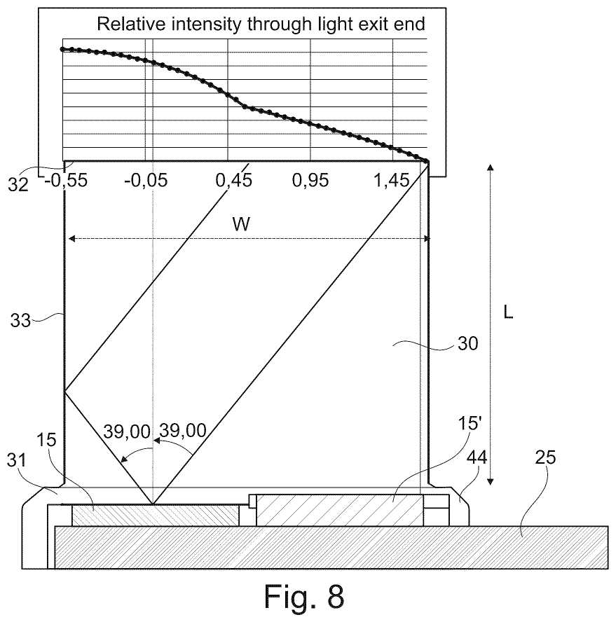

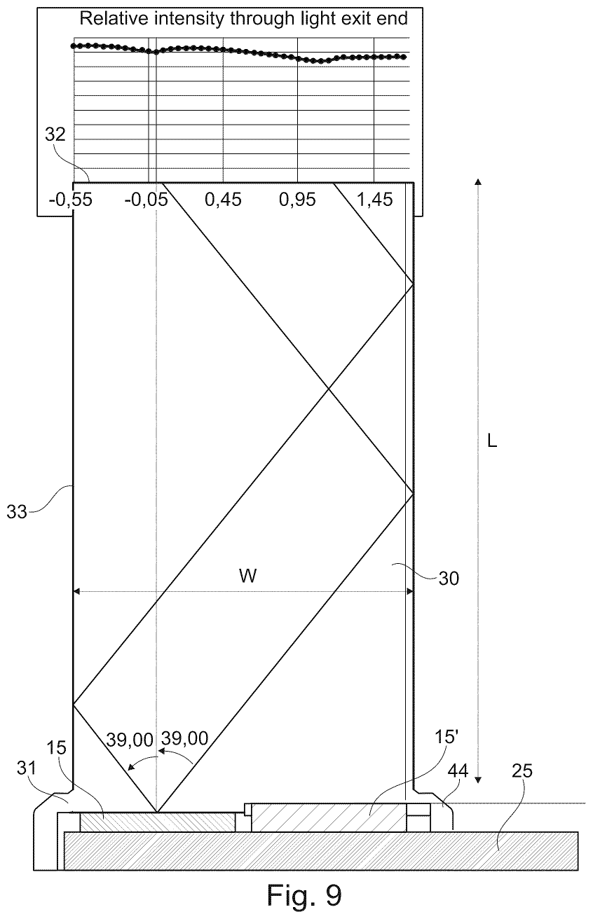

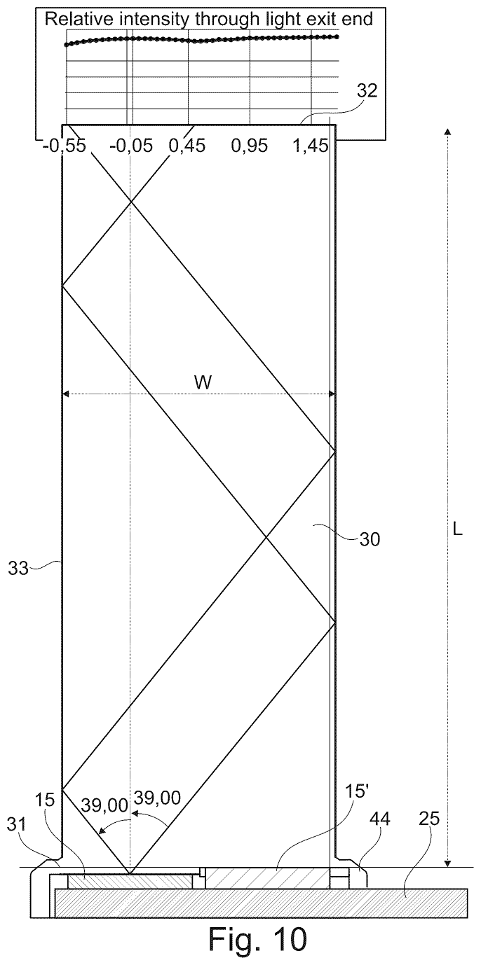

FIG. 9 shows the distribution of light intensity for a 4 mm long light guide (ratio 1:2, form factor 2), which provides an almost homogeneous light intensity distribution. Even a 2 mm long light guide 30 may be used in the distal tip 10 of an endoscope 1 even though the light intensity distribution will not be homogeneous (shown in FIG. 8). However, if the length of the light guide 30 is only 1 mm, as shown in FIG. 7, it is seen that there will be parts of the light exit end 32 which receives a relatively small amount of light intensity from the one powered LED 15.

For any length of light guide 16 limited to fit within the dimensions of a tip housing 16 of an endoscope, the off-centered placement of the LEDs 15 in relation to a centered longitudinal axis of the light guide means that more light rays from an LED 15 will be reflected in a nearby side wall 33 of the light guide, and relatively fewer light rays will be reflected in an oppositely placed side wall further away from the LED. This results in a non-symmetric angular distribution of light rays exiting the light exit end 32 of the light guide 30.

This non-symmetric angular distribution of light from each of at least two LEDs 30 having different colors means that in some angles of the illumination light one color may be more dominant than others. When the illumination light is reflected in tissue during an endoscopic procedure, it may be possible to see colored reflections caused by the non-symmetric distribution. Such reflections of different colors may be disturbing to the endoscope user, even though the use of the light guide will reduce the colored reflections.

Increasing the length of the light guide 30 will reduce the risk of such colored reflections. However, in practice it may be necessary to limit the length of the light guide to maybe 12 mm or less. Often a maximum length may be 15 mm. This limitation may be caused by a preference to have the light guide and the LEDs inside the tip housing 16, and not extending proximally of the tip housing 16. Thereby, it is simpler to seal off the tip housing in a watertight manner. There is an advantage in having the non-bendable stiff part distal to the bending section 20 as short as possible. I.e., the distal stiff part, comprising the distal end bending segment and the tip housing and any intermediate connector or transition part, should be as short as possible in a proximal to distal direction, as this will provide the best possible maneuverability of the endoscope 1.

It has been found that adding a diffuser 34 as shown in FIG. 6, close to or distal to the light exit end 32, can further reduce or remove the risk of getting colored reflections. A diffuser 34 may be referred to as a light diffuser or an optical diffuser. A diffuser has the effect of scattering the light. A diffuser may here be a transmissive diffuser, i.e., diffusing light passing through the diffuser. A diffuser 34 may be a randomized or stochastic microstructure which scatters all incoming light rays. The diffuser 34 may be a white diffusing glass or polymer made from a white translucent material. The diffuser 34 may, alternatively, be a transparent material provided with a microstructure or nanostructure surface. The transparent material may be a glass or a polymer, and the microstructure surface may be provided as a ground surface by sandblasting, or as part of the molding process. The diffuser may, alternatively, be made by a holographic etching on a transparent material. A diffuser may also be a combination of these alternatives and may also be made in other ways. Molding of a microtextured window is disclosed in commonly-owned U.S. Pat. No. 11,766,163.

The diffuser 34 may scatter light transmitted through the diffuser in a Lambertian scattering pattern. In Lambertian scattering the radiance of the scattered light is independent of the angle of view. The diffuser 34 may also scatter light to achieve a gaussian light intensity distribution. Also, other light intensity distributions on the distal side of the diffuser are possible.

The diffuser 34 may be a separate component, e.g., in the form of a film, or a diffusing pattern imprinted or molded at either the light exit end 32 of the light guide or at an inner or outer surface of the light emitter window 14, e.g., a microstructure made by a laser. The diffuser 34 may also be a layer of particles within the light guide material at or close to the light exit end 32, or within the light emitter window 14. The particles may be blended with the polymer material forming the light guide or the light emitter window, and the particle blend may then be injected during the molding process forming them.

In one example, the diffuser 34 is provided at a surface of the light emitter window or at the light exit end. In a further example, the light guide and the light emitter window 14 is one integrated part e.g., molded in one process or fused together, and the diffuser is provided at the external surface of the light emitter window 14. This may be by an imprinted or a molded pattern with a light diffusing effect on the external surface. Both examples reduce the number of components to be provided and assembled. Therefore, manufacturing cost can be reduced. The pattern can be etched on the mold's surface and formed onto to the molded part when the polymer is injected. Alternatively, a diffuser film layer can be placed in the mold and then the polymer can be injected, at which time the polymer fuses with the diffuser film layer. Molding of the light guide and the light emitter window may be as disclosed in commonly-owned U.S. Pat. No. 12,053,152. The light guide can also be adhesively bonded to the light emitter window.

It has been found in experiments that the combination of the one light guide 30 and the diffuser 34 makes it possible to avoid the colored reflections into the camera of an endoscope having at least two LEDs of different colors placed in the distal tip for illumination of a target to be observed.

The diffuser 34 may be designed to have a scattering angle corresponding to or being larger than the field of view of the camera 11. The scattering angle may be defined by the angle at which the light intensity is half the maximum light intensity. The maximum light intensity is often in the direction extending in parallel to the optical axis OA of the camera 11. The scattering angle may also be designed according to the intended use of the endoscope 1, thereby utilizing the light emitted from the LEDs in the most optimal way. A bronchoscope is mostly used in a tube-like structure and a relatively small scattering angle may be preferred. A cystoscope is mostly used in a spherical cavity and a relatively large scattering angle may be preferred. The scattering angle of a diffuser may be adjusted by varying particle size, or haze, or other parameters used to obtain the diffusive effect.

The diffuser 34 may be designed to cover the light exit end 32 of the light guide 30. If the light emitter window 14 is between the diffuser 34 and the light exit end 32, e.g., when the diffuser is on the external side of the light emitter window 14, the diffuser may be slightly larger than the light exit end of the light guide. Thereby, it may be avoided that light exiting the light exit end 32 close to the edge between the light exit end 32 and the side walls 33 of the light guide 30, can get around and bypass the diffuser 34.

The following items are further variations and examples of the embodiments described with reference to the figures.

1. An endoscope comprising a distal tip, including a camera with an optical axis, and a light emitter, the light emitter comprising: a first light emitting diode (LED) configured to emit light with a first spectral distribution; a second LED configured to emit light with a second spectral distribution which is different from the first spectral distribution; a light guide with a length, and a width transverse to the length, the light guide comprising a light entry end and a light exit end configured such that light passes the light guide in the length direction; and a diffuser arranged transverse to the optical axis; wherein the first and the second LED are arranged to emit light into the light entry end, and the diffuser is arranged such that light passing the light exit end also passes the diffuser.

2. The endoscope according to item 1, wherein the length of the light guide is equal to, or larger than the width of the light guide, or the length of the light guide is at least a factor of 2 larger than the width.

3. The endoscope according to item 1 or 2, wherein the distal tip comprises a light emitter window arranged such that light from the light exit end passes through the light emitter window, and where the light emitter window has an inner surface facing the light guide and an oppositely placed external surface.

4. The endoscope according to item 3, wherein the diffuser is provided at the inner surface or the external surface of the light emitter window or at the light exit end of the light guide.

5. The endoscope according to item 3 or 4, wherein the light guide and the light emitter window is one integrated part, where the light guide extends from the inner surface of the window, and the diffuser is placed at the external surface of the light emitter window.

6. The endoscope according to any one of the previous items, wherein the light emitter comprises a third LED configured to emit light with a third spectral distribution which is different from the first and second spectral distribution.

7. The endoscope according to item 6, wherein the light emitter comprises a fourth LED configured to emit light with a fourth spectral distribution which is different from the first, second and third spectral distributions.

8. The endoscope according to item 6 or 7, wherein the LEDs are arranged at the light entry end of the light guide in a 2×2 matrix.

9. The endoscope according to any one of the previous items, wherein the first LED has a peak of light emitted in the range 405-425 nm.

10. The endoscope according to any one of the previous items, wherein the second LED has a peak of light emitted in the range 520-540 nm.

11. The endoscope according to any one of the previous items, wherein the second LED, or the third LED according to item 6, has a peak of light emitted in the range 550-670 nm.

12. The endoscope according to any one of the previous items, wherein the length of the light guide is at maximum a factor of 8 larger than the width, or the length of the light guide is at maximum a factor of 6 larger than the width.

13. The endoscope according to any one of the previous items, wherein the power supply to at least the first and the second LEDs, is controllable for each LED separately, such that light intensity from each of at least the first and the second LEDs can be adjusted independently.

14. The endoscope according to any one of the previous items, wherein the distal tip comprises a housing with a support for positioning the camera, where the support is non-transparent to prevent stray-light into the image sensor.

15. A system comprising an endoscope according to any one of items 1-14, a monitor and a control unit.

### LIST OF REFERENCES

1 endoscope
2 handle
3 insertion cord
4 electrical cable with plug
5 main tube
6 working channel
7 electrical wires
10 distal tip
11 camera
12 camera window
13 light emitter
14 light emitter window
15 first LED
15′ second LED
15′ third LED
16 distal tip house
17 image sensor
18 lens stack
19 flexible printed circuit board
20 bending section
21 cleaning nozzle
22 waterjet nozzle
25 Printed Circuit Board for LEDs (PCB)
26 distal working channel opening
27 segment
28 hinge
30 Light guide
31 light entry end
32 light exit end
33 side wall
34 diffuser
36 support wall working channel tube
37 support wall media tubes
38 support structure for camera
40 screen
41 monitor
42 control unit
43 visualization system
44 rim
46 bending lever
OA optical axis
L length of light guide
W width of light guide

What is claimed is:

1. An endoscope comprising:
a distal tip including a light emitter and a camera with an optical axis, the light emitter comprising:
a light guide comprising a light entry end and a light exit end;
a first light emitting diode (LED) configured to emit light with a first spectral distribution, the first LED positioned to emit the light into the light entry end of the light guide;
a second LED configured to emit light with a second spectral distribution different from the first spectral distribution, the second LED positioned to emit the light into the light entry end of the light guide; and
a diffuser arranged transverse to the optical axis distally of the light entry end of the light guide such that the lights emitted by the first LED and the second LED pass through the light guide and also through the diffuser.

2. The endoscope of claim 1, wherein the distal tip comprises a light emitter window, wherein the light guide and the light emitter window are comprised in a one-piece part such that light from the light exit end passes through the light emitter window, wherein the light emitter window comprises an external surface facing away from the light guide, and wherein the diffuser is provided at the external surface of the light emitter window.

3. The endoscope of claim 2, wherein the diffuser is formed at, and forms, the external surface during molding of the one-piece part.

4. The endoscope of claim 1, wherein the distal tip comprises a light emitter window, wherein the light guide and the light emitter window are molded in a one-piece part with the light exit end of the light guide contiguous to an inner surface of the light emitter window without a seam surface therebetween, wherein the light emitter window comprises an external surface opposite the inner surface and facing distally away from the light guide, and wherein the diffuser is provided at the external surface of the light emitter window.

5. The endoscope of claim 1, wherein the distal tip comprises a light emitter window positioned distally of the light guide such that light from the light exit end passes through the light emitter window, wherein the light emitter window comprises an external surface facing away from the light guide and an inner surface opposite the external surface, and wherein the diffuser is provided at the light emitter window or at the light exit end of the light guide.

6. The endoscope of claim 5, wherein the diffuser is formed at the inner surface of the light emitter window or at the light exit end of the light guide.

7. The endoscope of claim 5, wherein the diffuser is applied at the external surface of the light emitter window.

8. The endoscope of claim 5, wherein the diffuser is applied at the inner surface of the light emitter window or the light exit end of the light guide.

9. The endoscope of claim 1, wherein the light emitter comprises a third LED configured to emit light with a third spectral distribution which is different from the first and second spectral distributions.

10. The endoscope of claim 9, wherein the light emitter comprises a fourth LED configured to emit light with a fourth spectral distribution which is different from the first, second and third spectral distributions.

11. The endoscope of claim 9, wherein the first, second, and third LEDs are arranged proximally of the light entry end of the light guide in a 2×2 matrix.

12. The endoscope of claim 9, wherein the first LED has a peak of light emitted in the range 405-425 nm, wherein the second LED has a peak of light emitted in the range 520-540 nm, and wherein the third LED has a peak of light emitted in the range 550-670 nm.

13. The endoscope of claim 1, wherein the first LED has a peak of light emitted in the range 405-425 nm.

14. The endoscope of claim 1, wherein the second LED has a peak of light emitted in the range 520-540 nm or in the range 550-670 nm.

15. The endoscope of claim 1, wherein the third LED has a peak of light emitted in the range 550-670 nm.

16. The endoscope of claim 1, further comprising a first cable electrically connected to the first LED and a second cable electrically connected to the second LED, the first cable and the second cable configured to supply power, respectively, to the first LED and the second LED separately such that a light intensity from each of the first and the second LEDs can be independently adjusted.

17. The endoscope of claim 1, wherein the distal tip comprises a housing including a support for positioning the camera, where the support is non-transparent to prevent stray-light into the image sensor.

18. The endoscope of claim 1, wherein the light guide comprises a length extending from the light entry end to the light exit end and a width transverse to the length, wherein the length of the light guide is at least twice the width but not larger than 8 times the width.

19. The endoscope of claim 1, wherein the light guide comprises a length extending from the light entry end to the light exit end and a width transverse to the length, wherein the length of the light guide is at maximum a factor of 8 larger than the width.

20. The endoscope of claim 4, wherein the length of the light guide is at maximum a factor of 6 larger than the width.

21. A system comprising the endoscope of claim 1, a monitor and a control unit.
